(19) United States
(12) Patent Application Publication
(10) Pub. No.: US 2018/0094280 A1
(43) Pub. Date: Apr. 5, 2018

(54) VECTOR FORMULATIONS

(71) Applicant: bluebird bio, Inc., Cambridge, MA (US)

(72) Inventors: Robert H. KUTNER, Allston, MA (US); Wesley GORMAN, Cambridge, MA (US); Francis J. PIERCIEY, Jr., Boston, MA (US)

(21) Appl. No.: 15/560,088

(22) PCT Filed: Mar. 18, 2016
(86) PCT No.: PCT/US2016/023271

§ 371 Date: Sep. 20, 2017

Related U.S. Application Data
(60) Provisional application No. 62/136,309, filed on Mar. 20, 2015.

(51) Int. Cl. C12N 15/861; A61K 39/21

(52) U.S. Cl. CPC C12N 15/8613; C12N 2710/10234; C12N 2740/15051; A61K 39/21

(57) ABSTRACT

The invention provides compositions and formulations and use of these compositions and formulations to preserve viral vectors. In particular, the particular embodiments relate to compositions and formulations of trehalose or derivatives thereof as suitable protectants for long-term storage of viral vectors and for both in vitro and in vivo applications

### CROSS REFERENCE TO RELATED APPLICATIONS

This application claims the benefit under 35 U.S.C. § 119(e) of U.S. Provisional Application No. 62/136,309, filed Mar. 20, 2015, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD_025_01WO_ST25.txt. The text file is 3 KB, was created on Mar. 18, 2016, and is being submitted electronically via EFS-Web, concurrent with the filing of the specification.

### BACKGROUND

### Technical Field

The invention relates generally to compositions and formulations to stabilize viral vectors. In particular, the present invention relates to compositions comprising trehalose or derivatives thereof for use in the long-term storage and stabilization of viral vectors for both in vitro and in vivo applications.

### Description of the Related Art

Gene therapy to combat a number of diseases has met with success recently. The use of retroviruses to deliver genes to mammalian cells is widespread, due to the retrovirus' ability to stably integrate into the host cell genome. A number of retroviral vectors have been used in gene therapy, including gammaretroviruses and lentiviruses.

Lentiviruses are complex retroviruses which, based on their higher level of complexity, can integrate into the genome of nonproliferating cells and modulate their life cycles, as in the course of latent infection. These viruses include HIV-1, HIV-2 and SIV, among others. Like other retroviruses, lentiviruses possess gag, pol and env genes which are flanked by two long terminal repeat (LTR) sequences. Each of these genes encodes multiple proteins, initially expressed as one precursor polyprotein. These proteins are important for the structure of the virus and the replication of its RNA genome. The 5′ and 3′ LTRs serve to promote transcription and polyadenylation of the virion RNAs.

Transduction potential and potency of lentivirus is influenced by various factors, including temperature, pH, freeze-and-thaw frequency and incubation conditions in growth media and serum components during vector production and storage. Lentivirus titer has been observed to decrease in a biphasic manner with increased freeze/thaw cycles and storage at higher temperatures (Kigashikawa and Chang 2001, Virology 280, 124-131). In order for gene therapy to be most effective, it is desirable to have retroviral vectors that maintain their potency.

### BRIEF SUMMARY

There is a need in the art for a stable aqueous composition comprising a viral vector, such as a lentiviral vector, which maintains infectious titer and/or expression of heterologous sequences when stored for various periods of time across a broad range of temperatures and is further suitable for in vitro or in vivo applications.

The present invention generally provides compositions and formulations for preservation of viral vectors. Embodiments provide aqueous compositions of trehalose or derivatives thereof as suitable protectants for long-term storage of viral vectors. Viral vectors stored in solutions containing particular concentrations of trehalose maintain infectious titer and/or expression of heterologous sequences in a broad range of temperatures from about −80° C. to about 20° C., throughout multiple freeze thaw cycles and over a broad range of time periods compared to viral vectors not stored in trehalose or derivatives thereof.

Thus, in certain embodiments, an aqueous composition is provided comprising: (a) a viral vector; (b) about 3% to about 15% trehalose or derivatives thereof by weight; and (c) a pharmaceutically acceptable diluent. In one embodiment, the viral vector is an adenoviral vector. In another embodiment, the viral vector is an adeno-associated viral vector. In yet another embodiment, the viral vector is a retroviral vector.

In one embodiment, the viral vector is a lentiviral vector. In another embodiment, the lentiviral vector is selected from the group consisting essentially of human immunodeficiency virus (HIV); visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, the lentiviral vector is HIV-1. In another embodiment, the lentiviral vector is HIV-2.

In one embodiment, the viral vector comprises an envelope polypeptide of a virus selected from the group consisting of: Avian leukosis virus (ALV), FIV, HIV, vesicular stomatitis virus (VSV), moloney murine leukemia virus (MoMLV), gibbon ape leukemia virus (GaLV), jaagsiekte sheep retrovirus (JSRV), lymphocytic choriomeningitis virus (LCMV), Human T-lymphotropic virus 1 (HTLV-1), visna-maedi virus (VMV), SARS-CoV, Chandipura virus, Marburg virus, Mokola virus, feline endogenous retrovirus (RD114), Ebola virus, Rabies virus, Ross River virus (RRV), Respiratory syncytia virus (RSV), Human parainfluenza virus type 3, Hepatitis C virus (HCV), Sendai virus, Sindbis virus, Semliki Forest virus (SFV), foul plague virus (FPV), influenza virus, Venezuelan equine encephalitis virus, and Lagos-bat virus. In a particular embodiment, the viral vector comprises the envelope polypeptide from VSV or RD114.

In one embodiment, the viral vector comprises a polynucleotide sequence encoding a chimeric antigen receptor (CAR) polypeptide, an engineered T cell receptor polypeptide, or a bi-specific T cell engager polypeptide.

In one embodiment, the CAR comprises: a) an extracellular domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1; b) a transmembrane domain derived from a polypeptide selected from the group consisting of: CD8α, CD4, CD28, CD45, PD1, CTLA-4, and CD152; c) one or more intracellular co-stimulatory signaling domains selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and d) a CD3ζ primary signaling domain.

In one embodiment, the extracellular domain of the CAR comprises an antibody or antigen binding fragment that binds the antigen. In another embodiment, the antibody or antigen binding fragment that binds the BCMA polypeptide is selected from the group consisting of: a Camel Ig, Ig NAR, Fab fragments, Fab′ fragments, F(ab)′₂ fragments, F(ab)′₃ fragments, Fv, single chain Fv antibody (“scFv”), bis-scFv, (scFv)₂, minibody, diabody, triabody, tetrabody, disulfide stabilized Fv protein (“dsFv”), and single-domain antibody (sdAb, Nanobody). In another embodiment, the antibody is a human antibody, a murine antibody, or a humanized antibody.

In one embodiment, the transmembrane domain of the CAR is derived from CD8α.

In one embodiment, the one or more intracellular co-stimulatory signaling domains of the CAR is selected from the group consisting of: CD28, CD134, and CD137. In another embodiment, the CAR comprises two or more intracellular co-stimulatory signaling domains selected from the group consisting of: CD28, CD134, and CD137. In a particular embodiment, the one or more intracellular co-stimulatory signaling domains of the CAR is CD28. In a particular embodiment, the one or more intracellular co-stimulatory signaling domains of the CAR is CD134. In a particular embodiment, the one or more intracellular co-stimulatory signaling domains of the CAR is CD137.

In one embodiment, the CAR further comprises a hinge region polypeptide. In another embodiment, the hinge region polypeptide comprises a hinge region of CD8α.

In one embodiment, the CAR further comprises a spacer region polypeptide. In another embodiment, the spacer region polypeptide comprises CH2 and CH3 regions of IgG1.

In one embodiment, the CAR further comprises a signal peptide. In another embodiment, the signal peptide comprises an IgG1 heavy chain signal polypeptide or a CD8a signal polypeptide.

In one embodiment, the viral vector comprises a polynucleotide sequence encoding a homing endonuclease, a transcription activator-like effector nuclease (TALEN), a zinc finger nuclease (ZFN), a Type II clustered regularly interspaced short palindromic repeats (CRISPR) associated (Cas9) nuclease, or a megaTAL nuclease.

In one embodiment, the viral vector comprises a polynucleotide sequence encoding a β-globin polypeptide.

In one embodiment, the viral vector comprises a polynucleotide sequence encoding an ABCD1 polypeptide.

In another embodiment, the aqueous composition comprises about 3%, 4%, 5%, 6%, 7%, 8%, 9%, 10%, 11%, 12%, 13%, 14% or 15% trehalose or derivatives thereof by weight. In one embodiment, the aqueous composition comprises about 3% trehalose or derivatives thereof by weight. In one embodiment, the aqueous composition comprises about 4% trehalose or derivatives thereof by weight. In one embodiment, the aqueous composition comprises about 5% trehalose or derivatives thereof by weight. In one embodiment, the aqueous composition comprises about 6% trehalose or derivatives thereof by weight. In one embodiment, the aqueous composition comprises about 7% trehalose or derivatives thereof by weight. In one embodiment, the aqueous composition comprises about 8% trehalose or derivatives thereof by weight. In one embodiment, the aqueous composition comprises about 9% trehalose or derivatives thereof by weight. In one embodiment, the aqueous composition comprises about 10% trehalose or derivatives thereof by weight. In one embodiment, the aqueous composition comprises about 11% trehalose or derivatives thereof by weight. In one embodiment, the aqueous composition comprises about 12% trehalose or derivatives thereof by weight. In one embodiment, the aqueous composition comprises about 13% trehalose or derivatives thereof by weight. In one embodiment, the aqueous composition comprises about 14% trehalose or derivatives thereof by weight. In one embodiment, the aqueous composition comprises about 15% trehalose or derivatives thereof by weight. In one embodiment, the composition comprises about 5% to about 15% trehalose or derivatives thereof by weight. In another embodiment, the composition comprises about 4% to about 12% trehalose or derivatives thereof by weight. In another embodiment, the composition comprises about 5% to about 10% trehalose or derivatives thereof by weight. In another embodiment, the composition comprises about 5% to about 9% trehalose or derivatives thereof by weight. In another embodiment, the composition comprises about 5% to about 8% trehalose or derivatives thereof by weight. In another embodiment, the composition comprises about 5% to about 7% trehalose or derivatives thereof by weight. In another embodiment, the composition comprises about 4% to about 6% trehalose or derivatives thereof by weight.

In one embodiment, the pharmaceutically acceptable diluent comprises a physiologically acceptable buffer. In another embodiment, the physiologically acceptable buffer is selected from the group consisting of: Hanks buffered saline solution (HBSS), Ringer's solution, Dulbecco's phosphate buffered saline (PBS), 5% dextrose in water (D5W), and physiologic saline (0.9% NaCl). In one embodiment, the pharmaceutically acceptable diluent comprises a physiologically acceptable cell culture medium. In another embodiment, the pharmaceutically acceptable cell culture medium is selected from the group consisting of: StemSpan-ACF, StemSpan-H3000, StemSpan-SFEM, Stemline II, StemPro 34, StemXVivo, Iscove's modified Dulbecco's medium (IMDM), Dulbecco's modified Eagle medium (DMEM), Roswell Park Memorial Institute medium (RPMI) 1640 medium, McCoy's 5A medium, minimum essential medium alpha medium (alpha-MEM), basal medium Eagle (BME), Fischer's medium, medium199, F-12K nutrient mixture medium (Kaighn's modification, F-12K), and X-vivo 20.

In another embodiment, the viral vector has a titer that is stable for more than one month when the composition is stored at about 20° C. In one embodiment, the viral vector has a titer that is stable for more than six months when the composition is stored at about 20° C. In another embodiment, the viral vector has a titer that is stable for more than one year when the composition is stored at about 20° C.

In one embodiment, the viral vector has a titer that is stable for more than one month when the composition is stored at about 10° C. In another embodiment, the viral vector has a titer that is stable for more than six months when the composition is stored at about 10° C. In another embodiment, the viral vector has a titer that is stable for more than one year when the composition is stored at about 10° C.

In one embodiment, the viral vector has a titer that is stable for more than one month when the composition is stored at about 4° C. In another embodiment, the viral vector has a titer that is stable for more than six months when the composition is stored at about 4° C. In another embodiment, the viral vector has a titer that is stable for more than one year when the composition is stored at about 4° C.

In one embodiment, the viral vector has a titer that is stable for more than one year when the composition is stored at about 0° C. In another embodiment, the viral vector has a titer that is stable for more than one year when the composition is stored at about −20° C. In another embodiment, the viral vector has a titer that is stable for more than one year when the composition is stored at about −60° C. In another embodiment, the viral vector has a titer that is stable for more than one year when the composition is stored at about −70° C. In another embodiment, the viral vector has a titer that is stable for more than one year when the composition is stored at about −80° C.

In another embodiment, the titer of the viral vector is stable for one or more freeze/thaw cycles. In another embodiment, the titer of the viral vector is stable for two or more freeze/thaw cycles. In another embodiment, the titer of the viral vector is stable for three or more freeze/thaw cycles.

In one embodiment, the composition is suitable for direct in vivo injection. In another embodiment, the composition is suitable for direct in vitro use.

In one embodiment, the composition is diluted at least 10× prior to direct in vivo injection or direct in vitro use. In another embodiment, the composition is diluted at least 50× prior to direct in vivo injection or direct in vitro use. In another embodiment, the composition is diluted at least 100× prior to direct in vivo injection or direct in vitro use. In another embodiment, the composition is diluted at least 200× prior to direct in vivo injection or direct in vitro use. In another embodiment, the composition is diluted at least 250× prior to direct in vivo injection or direct in vitro use.

In various embodiments, a method of treating a disease in a subject in need of treatment is contemplated, comprising administering to said subject a population of cells transduced with an aqueous composition described herein.

In one embodiment, a method of transducing a cell with an aqueous composition described herein is contemplated comprising introducing the aqueous composition into a population of cells. In one embodiment, the cell is a mammalian cell. In another embodiment, the cell is a human cell. In another embodiment, the cell is a stem cell or progenitor cell. In a further embodiment, the cell is a hematopoietic cell.

In one embodiment, the hematopoietic cell is a hematopoietic stem or progenitor cell or a cell that expresses CD34. In another embodiment, the hematopoietic cell is a lymphocyte. In yet another embodiment, the lymphocyte is a T lymphocyte.

In another embodiment, a method for stabilizing viral vectors is contemplated comprising: (a) preparing a composition comprising trehalose or derivatives thereof; and (b) adding a viral vector to the composition. In one embodiment, the viral vector has a titer that is stable when stored at a temperature range of about −80° C. to about 25° C. for at least one year. In another embodiment, the viral vector has a titer that is stable when stored at a temperature range of about −20° C. to about 18° C. for at least one year. In another embodiment, the viral vector has a titer that is stable when stored at a temperature range of about −20° C. to about 4° C. for at least one year. In a further embodiment, the viral vector has a titer that is stable when stored at a temperature range of about 4° C. to about 25° C. for at least one year. In yet another embodiment, the viral vector has a titer that is stable when stored at a temperature range of about 4° C. to about 18° C. for at least one year.

### BRIEF DESCRIPTION OF THE SEVERAL VIEWS OF THE DRAWINGS

FIG. 1 shows the effect of thawing on lentiviral vector titer examined in undiluted lentiviral vector compositions stored for 24 hours at 4° C. and after a single thaw when stored at −80° C. for 24 hours. FIG. 1 shows that the mean titer (TU/mL) was reduced in undiluted lentiviral vector compositions stored at −80° C. compared to undiluted lentiviral vector stored at 4° C.

FIG. 2 shows the long-term stability of lentiviral vectors stored undiluted in PBS or diluted in 5% trehalose/PBS examined over various temperatures for extended periods of storage. Titer was measured for lentiviral vectors stored for 4, 14, 28, 42, and 73 days at 4° C. and at −20° C. and −80° C. after single thaws. FIG. 2 shows that lentiviral vectors formulated in 5% trehalose show stable lentiviral titer across the temperatures tested for at least 73 days.

FIG. 3 shows lentiviral vector expression (RFP expression) examined by flow cytometry in cells transduced with 1.0E+08 TU/mL formulated in various concentrations of trehalose. Greater percentages of cells containing the lentiviral vector expressed RFP when stored for 24 hours at 4° C. and after a single thaw when stored at −80° C. for 24 hours in trehalose/PBS compared to cells containing the lentiviral vector stored undiluted in PBS.

FIG. 4 shows the change in mean titer (TU/mL) for the cell samples from FIG. 3 examined to determine which storage conditions provided the most protection for the lentiviral vector when stored at the lower temperature of −80° C. compared to when stored at 4° C. Cells containing the lentiviral vector stored undiluted in PBS had large negative Δ TU/mL values, indicating that the lentiviral vector did not retain stability once stored at −80° C. for 24 hours and then thawed compared to storage at 4° C. Cells containing the lentiviral vector stored in about 15% trehalose/PBS showed the largest positive Δ TU/mL values, indicating that the mean titers for the lentiviral vector actually increased after storage at −80° C. for 24 hours and then thawed compared to storage at 4° C.

FIG. 5 shows the titer of lentiviral vectors (Y-axis) plotted against the trehalose concentration (% by weight, X-axis) of the storage composition. The percentage of trehalose that yielded the highest preservation of the lentiviral vector with the lowest dilution at both 4° C. and −80° C. was about 15%.

FIG. 6 shows the titer of lentiviral vectors examined for samples stored at −80° C. in various concentrations of trehalose/PBS. For either concentrated product undiluted or diluted 1:2 in PBS, the preservative effect of trehalose was linear at the lower trehalose concentrations tested.

### DETAILED DESCRIPTION

### A. Overview

Gene therapy involves introducing a corrected gene of interest into a target cell population, typically using viral vectors. Viral vectors such as lentiviruses are affected negatively by storage conditions, such as storage at 4° C. for long periods of time or experiencing multiple freeze/thaw cycles after storage at lower temperatures such as −80° C. Lentivirus titer has been observed to decrease with increasing freeze/thaw cycles and increasing temperature. The present inventors have identified aqueous compositions that maintain the potency of viral vectors under various temperatures and time periods compared to viral vectors not stored in these aqueous compositions.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); and Harlow and Lane, Antibodies, (Cold Spring Harbor Laboratory Press, Cold Spring Harbor, N.Y., 1998).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

The term “substantially” or “essentially” refers to a quantity, level, value, number, frequency, percentage, dimension, size, amount, weight, length, or titer or other measure that is about 90%, 91%, 92%, 93%, 94%, 95%, 96%, 97%, 98%, or 99% or higher compared to a reference quantity, level, value, number, frequency, percentage, dimension, size, amount, weight, length, or titer or other measure.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “agent” or “compound” encompasses polynucleotides, polypeptides, and other organic and inorganic chemicals, including without limitation, all analogs and derivatives thereof.

In reference to chemicals, such as organic chemicals, “analog” or “derivative” relates to a chemical molecule that is similar to another chemical substance in structure and function, often differing structurally by a single element or group, but may differ by modification of more than one group (e.g., 2, 3, or 4 groups) if it retains the same function as the parental chemical. Such modifications are routine to persons skilled in the art, and include, for example, additional or substituted chemical moieties, such as esters or amides of an acid, protecting groups such as a benzyl group for an alcohol or thiol, and tert-butoxylcarbonyl groups for an amine. Also included are modifications to alkyl side chains, such as alkyl substitutions (e.g., methyl, dimethyl, ethyl, etc.), modifications to the level of saturation or unsaturation of side chains, and the addition of modified groups such as substituted phenyl and phenoxy. Derivatives may also include conjugates, such as biotin or avidin moieties, enzymes such as horseradish peroxidase and the like, and including radio-labeled, bioluminescent, chemoluminescent, or fluorescent moieties. Also, moieties may be added to the agents described herein to alter their pharmacokinetic properties, such as to increase half-life in vivo or ex vivo, or to increase their cell penetration properties, among other desirable properties. Also included are prodrugs, which are known to enhance numerous desirable qualities of pharmaceuticals (e.g., solubility, bioavailability, manufacturing, etc.) (see e.g., WO/2006/047476 for exemplary EP agonist prodrugs, which is incorporated by reference for its disclosure of such agonists).

To “preserve” refers to the use of a “protectant” or compound which can be included in a composition or formulation to essentially maintain the biological activity or integrity of a biological material. For example, the infectious titer of and/or expression of heterologous sequences in a viral vector can be preserved with trehalose. Examples of compounds to preserve biological materials include trehalose, derivatives of trehalose, glycerol, dimethyl sulfoxide, ethylene glycol, glucose, sucrose and maltose. Also, by “maintain,” or “preserve,” or “maintenance,” or “no change,” or “no substantial change,” or “no substantial decrease” refers generally to a physiological response that is comparable to a response caused by either vehicle, a control molecule/composition, or the response in a particular cell lineage. A comparable response is one that is not significantly different or measurable different from the reference response. In particular embodiments, a viral vector is preserved at a temperature when its titer is maintained for a duration of time at that temperature or where the titer decreases less than about 1%, less than about 2%, less than about 3%, less than about 4%, less than about 5%, less than about 10%, less than about 12%, less than about 15%, less than about 18%, less than about 20%, less than about 22%, or less than about 25%, or any intervening value.

A “stable” composition is one in which the viral vector therein substantially retains its infectious titer, vector particle to infectivity ratio, and/or expression of heterologous sequences upon storage at a temperature for a period of time. Techniques for measuring viral vector stability are described in the Examples and known in the art. Stability can be measured at a selected temperature for a selected time period. Stability may also be measured on top of the assay variability, which in particular embodiments is about 20%, about 25%, about 30%, or about 35%. Preferably, the composition is stable at about 20° C., at about 10° C., at about 4° C., at about 0° C., at about −5° C., at about −10° C., at about −20° C., at about −30° C., at about −40° C., at about −50° C., at about −60° C., at about −70° C., at about −80° C., or any temperature in between for a given time period. Preferably, the composition is stable for at least about 24 hours, at least about 1 month, at least about 6 months, at least about 1 year, at least about 2 years or any time period in between for a given temperature.

In particular embodiments, the viral vector is stable at a temperature when its infectious titer is maintained for a duration of time (e.g., hours, days, weeks, months, years) at that temperature or where the titer decreases less than about 1%, less than about 2%, less than about 3%, less than about 4%, less than about 5%, less than about 10%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, or less than about 35%, or any intervening value compared to the originally measured titer. In one embodiment, the viral vector is stable at a temperature when its infectious titer decreases less than about 15% to less than about 25%, or any intervening value compared to the originally measured titer.

In particular embodiments, the viral vector is stable at a temperature when the infectious titer is about 95%, about 94%, about 93%, about 92%, about 92%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, or about 65% of the original titer when measured for a duration of time (e.g., hours, days, weeks, months, years) at that temperature. In one embodiment, the titer of a viral vector is stable at a temperature when the infectious titer is about 85% to about 75%, or any intervening value of the original titer when measured for a duration of time at that temperature.

In particular embodiments, the viral vector is stable at a temperature when the vector particle to infectivity (P:I) ratio of the virus is within about 45%, about 50%, about 55%, about 60%, about 65%, about 70%, about 75%, about 80%, about 85%, about 90%, about 95%, or any intervening value of the original or first measured (P:I) of the virus. In one embodiment, the viral vector is stable at a temperature when the vector particle to infectivity (P:I) ratio of the virus is within about 50% or more of the original or first measured (P:I) of the virus.

One of skill in the art will understand that temperatures above and below the listed temperatures, and time periods shorter and longer than the listed time periods, may apply when measuring the stability of a composition. Furthermore, the composition is preferably stable following freezing (to, e.g., −80° C.) and thawing of the composition, e.g., for 1, 2, 3, 4, 5, or more freeze/thaw cycles.

### C. Trehalose and Derivatives

In various embodiments, compositions and formulations contemplated herein comprise a viral vector and trehalose or a derivative thereof.

Trehalose (also known as α,α-trehalose, α-D-glucopyranosyl α-D-glucopyranoside, mushroom sugar, mycose) is a naturally occurring disaccharide containing two D-glucose units in an α,α-1,1 linkage:

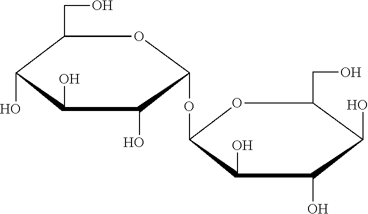

Trehalose can be found in plants, algae, fungi, yeasts, bacteria, insects and other invertebrates. It is cleaved by trehalase, a highly specific enzyme that is found in multiple forms in organisms that contain trehalose. It is not easily hydrolyzed by acid, and the glycosidic bond is not cleaved by α-glucosidase. This non-reducing sugar is characterized by its high degree of optical rotation and melting behavior. It is considered to be a very stable disaccharide. Isomers of trehalose include α,β (neotrehalose) and β,β (isotrehalose), although these isomers are rarely found in nature.

Modern food sources that contain trehalose include honey, mirin, sherries, many items made using yeast, commercially grown mushrooms and invertebrates such as lobster, crab and prawn. Trehalose can be isolated from seed plants such as sunflower.

Trehalose may have a number of roles in nature, including serving as an energy source during development, as a structural component or as a metabolic intermediate. In insects, it appears to provide the energy source for flight.

“Trehalose derivative” or “derivative of trehalose” includes a compound derived from trehalose by a chemical or physical process, wherein said compound does not contain a free carbonyl or anomeric carbon, the carbonyl carbon from the aldehyde or ketone group being involved in a glycosidic bond. Some examples of derivatives of trehalose are 2,3,2′,3′-tetra-O-Benzyl-6,6′-di-O-decanoyl-4,4′-bis-O(diphenylphosphono)-alpha,alpha trehalose, 6,6′-di-O-decanoyl-4,4′-di-O-phosphono alpha,alpha trehalose, 2,3,2′,3′-tetra-O-benzyl-4,4′-bis-O(dipheynlphosphono)alpha,alpha trehalose 6,6′, fatty acid ester.

### d. Compositions and Formulations

The compositions contemplated herein may comprise trehalose or derivatives thereof and viral vectors contemplated herein. In particular embodiments, compositions for storing and/or stabilizing viral vectors can be prepared as follows: a stock solution of trehalose or derivatives thereof is made by dissolving an appropriate amount of trehalose dehydrate in a physiologically acceptable buffer to give a stock solution of trehalose, for example, a stock solution of about 1M to about 2 M trehalose; then the viral vector can be formulated in the same or different physiologically acceptable buffer by diluting at various ratios with the stock solution of trehalose to yield a composition, e.g., an aqueous solution, comprising the viral vector at a particular final percentage of trehalose by weight.

Compositions include, but are not limited to pharmaceutical compositions. A “pharmaceutical composition” refers to a composition formulated in pharmaceutically-acceptable or physiologically-acceptable solution or buffer for administration to a cell or an animal, either alone, or in combination with one or more other modalities of therapy. It will also be understood that, if desired, compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Physiologically acceptable solutions” or “physiologically acceptable buffer” refer to aqueous solutions that are acceptable for use in humans or domestic animals. Exemplary physiologically acceptable buffers include, but are not limited to pyrogen-free water; isotonic saline (0.9% NaCl); Hanks buffered saline solution (HBSS); Ringer's solution; 5% dextrose in water (D5W); and physiologically buffered salts, e.g., Dulbecco's phosphate buffered saline (PBS).

In particular embodiments, the compositions contemplated herein comprise a buffer to maintain the pH of the medium at an optimal value for a viral vector. For example, the buffer can be a phosphate buffer or another buffer that will allow a physiological pH of about 7. In particular embodiments, the composition has a pH of about 7, about 7.1, about 7.2, about 7.3 or about 7.4. In one embodiment, the composition has a pH of about 7.4.

In particular embodiments, aqueous compositions for storing and/or stabilizing viral vectors can be prepared as follows: a stock solution of trehalose or derivatives thereof is made by dissolving an appropriate amount of trehalose dehydrate in phosphate-buffered saline (PBS, pH 7.4) to give a stock solution of trehalose/PBS of, for example, about 1M to about 2 M; then the viral vector can be formulated in PBS is diluted at various ratios with the stock solution of trehalose/PBS to yield an aqueous composition comprising the viral vector at a particular final percentage of trehalose by weight.

In one embodiment, a viral vector is formulated in an aqueous composition comprising about 3% trehalose, about 4% trehalose, about 5% trehalose, about 6% trehalose, about 7% trehalose, about 8% trehalose, about 9% trehalose, about 10% trehalose, about 11% trehalose, about 12% trehalose, about 13% trehalose, about 14% trehalose, about 15% trehalose, about 16% trehalose, about 17% trehalose, about 1% trehalose, about 19% trehalose, or about 20% trehalose, or any intervening concentration of trehalose. In particular embodiments, one of skill in the art could adjust the volumes of the viral vector, stock trehalose and physiologically acceptable buffer according to the total volume desired.

The compositions contemplated herein stabilize viral vectors at a wide range of temperatures for extended periods of time. In particular embodiments, a composition comprises a viral vector and an effective amount of trehalose to stabilize the viral vector at about 20° C., at about 10° C., at about 4° C., at about 0° C., at about −5° C., at about −10° C., at about −20° C., at about −30° C., at about −40° C., at about −50° C., at about −60° C., at about −70° C., at about −80° C., or any temperature in between for an extended time period. In certain embodiments, the viral vector is stable for at least about 24 hours, at least about 48 hours, at least about 72 hours, at least about 96 hours, at least about 1 week, at least about 2 weeks, at least about 3 weeks, at least about 1 month, at least about 2 months, at least about 3 months, at least about 4 months, at least about 5 months, at least about 6 months, at least about 1 year, at least about 2 years or more or any time period. One of skill in the art will understand that temperatures above and below the listed temperatures, and time periods shorter and longer than the listed time periods, may apply when measuring the stability of a composition in particular embodiments.

The compositions contemplated herein stabilize viral vectors after freezing and thawing the vectors. In one embodiment, the composition stabilizes viral vectors for 1, 2, 3, 4, 5, 6, 7, 8, 9, 10 or more freeze/thaw cycles at various temperatures disclosed herein, e.g., −20° C., −60° C., −70° C., −80° C., etc. In another embodiment, the composition stabilizes viral vectors for more than 11 freeze/thaw cycles. In another embodiment, the composition stabilizes viral vectors for more than 20 freeze/thaw cycles.

In various embodiments, the compositions comprise one or more antioxidants, such as sodium thiosulfate, ascorbic acid, citric acid, and sodium citrate. If an antioxidant is used, it can be present in an amount known to be useful in the art.

In some embodiments, the compositions comprise other components that may act as drying agents and/or osmoprotectants, such as methanol, ethanol, glycerol and DMSO. These components tend to reduce residual moisture or balance osmotic stresses in the preserved viral vector compositions, which may in some cases result in better storage capability. In certain embodiments, the compositions comprise protein, such as human serum albumin or bovine serum albumin.

In particular embodiments, compositions comprise an amount of trehalose effective to stabilize a viral vector. As used herein, the term “amount” refers to “an amount effective” or “an effective amount” of trehalose or a derivative thereof, to achieve a result.

In certain embodiments, pharmaceutical compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions contemplated herein may also be formulated for parenteral administration, e.g., intravascular (intravenous or intraarterial), intraperitoneal or intramuscular administration. An injectable pharmaceutical composition is preferably sterile.

The aqueous composition comprising viral vectors can be administered by direct injection to a cell, tissue, or organ of a subject in need of gene therapy, in vivo. In various other embodiments, cells are transduced in vitro or ex vivo with compositions contemplated herein comprising stable viral vectors, and optionally expanded ex vivo. The transduced cells are then administered to a subject in need of gene therapy.

### E. Viral Vectors

In various embodiments, the compositions contemplated herein comprise an effective amount of trehalose or a derivative thereof to store or stabilize a viral vector. Exemplary types of vectors that can be stabilized by the compositions contemplated herein include, but are not limited to adenovirus, adeno-associated virus, retrovirus, and the like.

1. Adenovirus

“Adenovirus vectors” refer to those constructs containing adenovirus sequences sufficient to (a) support packaging of the construct and (b) to express a polynucleotide that has been cloned therein in a sense or antisense orientation. A recombinant Adenovirus vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al., (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 mL of medium. Following stirring at 40 rpm, the cell viability is estimated with trypan blue. In another format, Fibra-Cel microcarriers (Bibby Sterlin, Stone, UK) (5 g/1) is employed as follows. A cell inoculum, resuspended in 5 mL of medium, is added to the carrier (50 mL) in a 250 mL Erlenmeyer flask and left stationary, with occasional agitation, for 1 to 4 h. The medium is then replaced with 50 mL of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the particular embodiments. The adenovirus may be of any of the 42 different known serotypes or subgroups A-F. Adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As stated above, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 109-1011 plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993).

2. Adeno-Associated Virus

AAV (Ridgeway, 1988; Hermonat & Muzyczka, 1984) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka & McLaughlin, 1988).

The AAV DNA is approximately 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat & Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs.

AAV is also a good choice of delivery vehicles due to its safety, i.e., genetically engineered (recombinant) does not integrate into the host genome. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

Other viral vectors may be employed as expression constructs for the delivery of oligonucleotide or polynucleotide sequences to a host cell. Vectors derived from viruses such as vaccinia virus (Ridgeway, 1988; Coupar et al., 1988), polioviruses and herpes viruses may be employed. They offer several attractive features for various mammalian cells (Friedmann, 1989; Ridgeway, 1988; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

3. Retrovirus

Retroviruses are a common tool for gene delivery (Miller, 2000, Nature. 357: 455-460). In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising MND promoter and encoding a CAR to a cell.

Retroviral vectors and more particularly lentiviral vectors may be used in practicing particular embodiments. Accordingly, the term “retrovirus” or “retroviral vector”, as used herein is meant to include “lentivirus” and “lentiviral vectors” respectively.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs.” The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR is composed of U3, R and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., 1995. J. of Virology, Vol. 69, No. 4; pp. 2101-2109. Several retroviral vectors use the minimal packaging signal (also referred to as the psi [Ψ] sequence) needed for encapsidation of the viral genome. Thus, as used herein, the terms “packaging sequence,” “packaging signal,” “psi” and the symbol “Ψ,” are used in reference to the non-coding sequence required for encapsidation of retroviral RNA strands during viral particle formation.

In various embodiments, vectors comprise modified 5′ LTR and/or 3′ LTRs. Either or both of the LTRs may comprise one or more modifications including, but not limited to, one or more deletions, insertions, or substitutions. Modifications of the 3′

LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included in particular embodiments.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

As used herein, the term “FLAP element” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector comprises a FLAP element isolated from HIV-1.

In one embodiment, retroviral or lentiviral transfer vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE). Generally, the RNA export element is placed within the 3′ UTR of a gene, and can be inserted as one or multiple copies.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular embodiments, vectors comprise a posttranscriptional regulatory element such as a WPRE or HPRE.

In particular embodiments, vectors lack or do not comprise a posttranscriptional regulatory element such as a WPRE or HPRE because in some instances these elements increase the risk of cellular transformation and/or do not substantially or significantly increase the amount of mRNA transcript or increase mRNA stability. Therefore, in some embodiments, vectors lack or do not comprise a WPRE or HPRE as an added safety measure.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “poly(A) site” or “poly(A) sequence” as used herein denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Efficient polyadenylation of the recombinant transcript is desirable as transcripts lacking a poly(A) tail are unstable and are rapidly degraded. Illustrative examples of poly(A) signals that can be used in a vector, include an ideal poly(A) sequence (e.g., AATAAA, ATTAAA, AGTAAA), a bovine growth hormone poly(A) sequence (BGHpA), a rabbit β-globin poly(A) sequence (rβgpA), or another suitable heterologous or endogenous poly(A) sequence known in the art.

In certain embodiments, a retroviral or lentiviral vector further comprises one or more insulator elements. Insulators elements may contribute to protecting lentivirus-expressed sequences, e.g., therapeutic polypeptides, from integration site effects, which may be mediated by cis-acting elements present in genomic DNA and lead to deregulated expression of transferred sequences (i.e., position effect; see, e.g., Burgess-Beusse et al., 2002, Proc. Natl. Acad. Sci., USA, 99:16433; and Zhan et al., 2001, Hum. Genet., 109:471). In some embodiments, transfer vectors comprise one or more insulator elements at the 3′ LTR and upon integration of the provirus into the host genome, the provirus comprises the one or more insulators at both the 5′ LTR and 3′ LTR, by virtue of duplicating the 3′ LTR. Suitable insulators for use in particular embodiments include, but are not limited to, the chicken β-globin insulator (see Chung et al., 1993. Cell 74:505; Chung et al., 1997. PNAS 94:575; and Bell et al., 1999. Cell 98:387, incorporated by reference herein). Examples of insulator elements include, but are not limited to, an insulator from a β-globin locus, such as chicken HS4.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid.

In one embodiment, a vector comprises at least one modified or unmodified retroviral LTR, e.g., lentiviral LTR, a β-globin promoter and a β-globin locus control region (LCR) operably linked to a polynucleotide of interest, e.g., encoding a globin polypeptide. Suitable modifications of the LTRs include, but are not limited to: replacement of the 5′ LTR is with a heterologous promoter, e.g., cytomegalovirus (CMV) promoter, a Rous Sarcoma Virus (RSV) promoter, a thymidine kinase promoter, or a Simian Virus 40 (SV40) promoter; and one or more modifications, additions, and/or deletions of a 3′ LTR as discussed elsewhere herein.

In a particular embodiment, erythroid specific expression of a polynucleotide is achieved using a human β-globin promoter, a β-globin LCR that comprises one or more of DNAase I hypersensitive sites 2, 3 and 4 from the human β-globin LCR, and/or a human β-globin 3′ enhancer element.

In various embodiments, a vector comprises one or more elements selected from the group consisting of: a Psi packaging sequence (Ψ⁺), a central polypurine tract/DNA flap (cPPT/FLAP), a retroviral export element, a posttranscriptional regulatory element, one or more insulator elements, a polyadenylation sequence, a selectable marker, and a cell suicide gene, as discussed elsewhere herein.

In various embodiments, the vectors comprise a promoter operably in hematopoietic cell operably linked to a gene encoding a polypeptide that provides therapy for hemoglobinopathies. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences).

In one embodiment, a vector comprises a left (5′) retroviral LTR, a Psi packaging sequence (Ψ⁺), central polypurine tract/DNA flap (cPPT/FLAP), a retroviral export element, a β-globin promoter, a β-globin locus control region (LCR), and optionally a 3′ β-globin enhancer operably linked to a polynucleotide of interest, and a right (3′) retroviral LTR that comprises one or more insulator elements, or a polyadenylation sequence.

In particular embodiment, a vector is a lentiviral vector that comprises a left (5′) HIV-1 LTR, a Psi packaging sequence (Ψ⁺), an HIV-1 central polypurine tract/DNA flap (cPPT/FLAP), a rev response element (RRE), a β-globin promoter, a β-globin locus control region (LCR), and optionally a 3′ β-globin enhancer operably linked to a polynucleotide of interest, and a right (3′) retroviral LTR that comprises one or more insulator elements, and a rabbit β-globin poly(A) sequence (rβgpA).

In various embodiments, the vectors comprise a promoter operably in a microglial cell operably linked to a gene encoding a polypeptide that provides therapy for adrenoleukodystrophies and/or adrenomyeloneuropathies. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences).

In a particular embodiment, the vector comprises a left (5′) retroviral LTR; a central polypurine tract/DNA flap (cPPT/FLAP); a retroviral export element; a promoter active in a microglial cell, operably linked to a polynucleotide encoding an ATP-binding cassette, sub-family D, member 1 (ABCD1) polypeptide; and a right (3′) retroviral LTR.

In a certain embodiment, the vector is a lentiviral vector comprising: a left (5′) HIV-1 LTR; a Psi (Ψ) packaging signal; a cPPT/FLAP; an RRE; a MND promoter, operably linked to a polynucleotide encoding a human ABCD1 polypeptide; a right (3′) self-inactivating (SIN) HIV-1 LTR; and a rabbit β-globin polyadenylation sequence.

In various embodiments, the vectors comprise a promoter operably linked to a polynucleotide encoding a CAR polypeptide. The vectors may have one or more LTRs, wherein either LTR comprises one or more modifications, such as one or more nucleotide substitutions, additions, or deletions. The vectors may further comprise one or more accessory elements to increase transduction efficiency (e.g., a cPPT/FLAP), viral packaging (e.g., a Psi (Ψ) packaging signal, RRE), and/or other elements that increase therapeutic gene expression (e.g., poly (A) sequences), and may optionally comprise a WPRE or HPRE.

In a particular embodiment, the transfer vector comprises a left (5′) retroviral LTR; a central polypurine tract/DNA flap (cPPT/FLAP); a retroviral export element; an MND promoter operably linked to a polynucleotide encoding CAR polypeptide contemplated herein; and a right (3′) retroviral LTR; and optionally a WPRE or HPRE.

In a particular embodiment, the transfer vector comprises a left (5′) retroviral LTR; a retroviral export element; a MND promoter operably linked to a polynucleotide encoding CAR polypeptide contemplated herein; a right (3′) retroviral LTR; and a poly (A) sequence; and optionally a WPRE or HPRE. In another particular embodiment, a lentiviral vector comprises: a left (5′) LTR; a cPPT/FLAP; an RRE; an MND promoter operably linked to a polynucleotide encoding CAR polypeptide contemplated herein; a right (3′) LTR; and a polyadenylation sequence; and optionally a WPRE or HPRE. In a certain embodiment, a lentiviral vector comprises: a left (5′) HIV-1 LTR; a

Psi (Ψ) packaging signal; a cPPT/FLAP; an RRE; an MND promoter operably linked to a polynucleotide encoding CAR polypeptide contemplated herein; a right (3′) self-inactivating (SIN) HIV-1 LTR; and a rabbit β-globin polyadenylation sequence; and optionally a WPRE or HPRE.

In another embodiment, a vector comprises: at least one LTR; a central polypurine tract/DNA flap (cPPT/FLAP); a retroviral export element; and an MND promoter operably linked to a polynucleotide encoding CAR polypeptide contemplated herein; and optionally a WPRE or HPRE.

In a particular embodiment, a vector comprises at least one LTR; a cPPT/FLAP; an RRE; an MND promoter operably linked to a polynucleotide encoding CAR polypeptide contemplated herein; and a polyadenylation sequence; and optionally a WPRE or HPRE.

In a certain embodiment, a vector comprises at least one SIN HIV-1 LTR; a Psi (Ψ) packaging signal; a cPPT/FLAP; an RRE; an MND promoter operably linked to a polynucleotide encoding CAR polypeptide contemplated herein; and a rabbit β-globin polyadenylation sequence; and optionally a WPRE or HPRE.

In a particular embodiment, a vector comprises a polynucleotide sequence encoding a homing endonuclease, a transcription activator-like effector nuclease (TALEN), a zinc finger nuclease (ZFN), a Type II clustered regularly interspaced short palindromic repeats (CRISPR) associated (Cas9) nuclease, or a megaTAL nuclease.

The skilled artisan would appreciate that many other different embodiments can be fashioned from the existing embodiments.

Recombinant viruses with titers of several millions of transducing units per milliliter (TU/mL) can be generated by techniques known in the art. After ultracentrifugation concentrated stocks of about 10⁸ TU/mL, 10⁹ TU/mL, 10¹⁰ TU/mL, 10¹¹ TU/mL, 10¹² TU/mL, or about 10¹³ TU/mL can be obtained.

Viruses may be used to infect cells in vivo, ex vivo, or in vitro using techniques well known in the art. For example, when cells, for instance CD34⁺ cells, dendritic cells, peripheral blood cells or stem cells are transduced ex vivo, the vector particles may be incubated with the cells using a dose generally in the order of between 1 to 50 multiplicities of infection (MOI) which also corresponds to 1×10⁵ to 50×10⁵ transducing units of the viral vector per 10⁵ cells. This, of course, includes amount of vector corresponding to 1, 2, 3, 4, 5, 6, 7, 8, 9, 10, 15, 20, 25, 30, 35, 40, 45, and 50 MOI.

Viruses may also be delivered to a subject in vivo, by direct injection to the cell, tissue, or organ in need of therapy. Direct injection requires on the order of between 1 to 50 multiplicities of infection (MOI) which also corresponds to 1×10⁵ to 50×10⁵ transducing units of the viral vector per 10⁵ cells.

Viruses may also be delivered according to viral titer (TU/mL), which can be measured, for example, by using a commercially available p24 titer assay, which is an ELISA against the p24 viral coat protein. The following formula can be used to calculate the pg/mL of p24: there are approximately 2000 molecules of p24 per physical particle (PP) of lentivirus: (2×10³)×(24×103 Da of p24 per PP), 48×10⁶/Avogadro=(48×10⁶)/(6×10²³)=8×10⁻¹⁷ g of p24 per PP, approximately 1 PP per 1×10-¹⁶ g of p24, 1×10⁴ PP per pg of p24. A reasonably well packaged, VSV-G pseudotyped lentiviral vector will have an infectivity index in the range of 1 TU per 1000 physical particles (PP) to 1 TU per 100 PP (or less). Thus, the range is approximately 10 to 100 TU/pg of p24. It is through this conversion that TU/mL is obtained.

Based on previous experience, the amount of virus directly injected is determined by total TU and can vary based on both the volume that could be feasibly injected to the site and the type of tissue to be injected. For example, a brain injection site may only allow for a very small volume of virus to be injected, so a high titer prep would be preferred, a TU of about 1×10⁶ to 1×10⁷, about 1×10⁶ to 1×10⁸, 1×10⁶ to 1×10⁹, about 1×10⁷ to 1×10¹⁰, 1×10⁸ to 1×10¹¹, about 1×10⁸ to 1×10¹², or about 1×10¹⁰ to 1×10¹² or more per injection could be used. However, a systemic delivery could accommodate a much larger TU, a load of 1×10⁸, 1×10⁹, 1×10¹⁰, 1×10¹¹, 1×10¹², 1×10¹³, 1×10¹⁴, or 1×10¹⁵, could be delivered.

### F. Engineered T Cell Receptors

In various embodiments, the compositions contemplated herein comprise an effective amount of trehalose or a derivative thereof to store or stabilize a viral vector encoding an engineered TCR.

Naturally occurring T cell receptors comprise two subunits, an α-subunit and a β-subunit, each of which is a unique protein produced by a recombination event in each T cell's genome. Libraries of TCRs may be screened for their selectivity to particular target antigens. In this manner, natural TCRs, which have a high-avidity and reactivity toward target antigens may be selected, cloned, and subsequently introduced into a population of T cells used for adoptive immunotherapy.

The nucleic acids encoding engineered TCRs are preferably isolated from their natural context in a (naturally-occurring) chromosome of a T cell, and can be incorporated into suitable vectors as described elsewhere herein. Both the nucleic acids and the vectors comprising them usefully can be transferred into a cell, which cell is preferably a T cell. The essential embodiment of the engineered TCRs is that it has high avidity for a tumor antigen presented by a major histocompatibility complex (MHC) or similar immunological component. In contrast to engineered TCRs, CARs are engineered to bind target antigens in an MHC independent manner.

The protein encoded by the inventive nucleic acids can be expressed with additional polypeptides attached to the amino-terminal or carboxyl-terminal portion of the inventive α-chain or β-chain of a TCR so long as the attached additional polypeptide does not interfere with the ability of the α-chain or β-chain to form a functional T cell receptor and the MHC dependent antigen recognition.

Antigens that are recognized by the engineered TCRs contemplated herein include, but are not limited to cancer antigens, including antigens on both hematological cancers and solid tumors. Illustrative antigens include, but are not limited to alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1.

### G. Chimeric Antigen Receptors

In various embodiments, the compositions contemplated herein comprise an effective amount of trehalose or a derivative thereof to store or stabilize a viral vector encoding a chimeric antigen receptor (CAR). CARs are molecules that combine antibody-based specificity for a target antigen (e.g., tumor antigen) with a T cell receptor-activating intracellular domain to generate a chimeric protein that exhibits a specific anti-tumor cellular immune activity. As used herein, the term, “chimeric,” describes being composed of parts of different proteins or DNAs from different origins.

Vectors contemplated herein comprise a promoter and a polynucleotide encoding a CAR. The CARs contemplated herein comprise an extracellular domain that binds to a specific target antigen (also referred to as a binding domain or antigen-specific binding domain), a transmembrane domain and an intracellular signaling domain. Engagement of the antigen binding domain of the CAR with its target antigen on the surface of a target cell results in clustering of the CAR and delivers an activation stimulus to the CAR-containing cell. The main characteristic of CARS are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In particular embodiments, a CAR comprises an extracellular binding domain including but not limited to an antibody or antigen binding fragment thereof, a tethered ligand, or the extracellular domain of a co-receptor, that specifically binds a target antigen selected from the group consisting of: alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1; one or more hinge domains or spacer domains; a transmembrane domain including, but not limited to, transmembrane domains from alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, and PD-1; one or more intracellular co-stimulatory signaling domains including but not limited to intracellular co-stimulatory signaling domains from TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from CD3ζ or FcRγ.

Exemplary binding domains include, but are not limited to antibody or antigen binding fragment thereof that specifically bind a target antigen. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell. Antibodies include antigen binding fragments thereof, such as Camel Ig, Ig NAR, Fab fragments, Fab′ fragments, F(ab)′2 fragments, F(ab)′3 fragments, Fv, single chain Fv proteins (“scFv”), bis-scFv, (scFv)2, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, Ill.); Kuby, J., Immunology, 3rd Ed., W. H. Freeman & Co., New York, 1997.

A “target antigen” or “target antigen of interest” is an antigen that a binding domain of a CAR contemplated herein, is designed to bind. In particular embodiments, the target antigen is an epitope of a peptide, lipid, polysaccharide, or nucleic acid, to which the binding domain specifically binds. In a preferred embodiment, the antigen is an epitope of an alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, VEGFR2, and WT-1 polypeptide.

In certain preferred embodiments, the antibody or fragment is humanized (such as a humanized monoclonal antibody) that specifically binds to a surface protein on a tumor cell. A “humanized” antibody is an immunoglobulin including a human framework region and one or more CDRs from a non-human (for example a mouse, rat, or synthetic) immunoglobulin.

In certain embodiments, the CARs contemplated herein may comprise linker residues between the various domains, e.g., between V_{H} and V_{L} domains, added for appropriate spacing and conformation of the molecule. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO: 1); TGEKP (SEQ ID NO: 2) (see, e.g., Liu et al., PNAS 5525-5530 (1997)); GGRR (SEQ ID NO: 3) (Pomerantz et al. 1995, supra); (GGGGS)ₙ wherein=1, 2, 3, 4 or 5 (SEQ ID NO: 4) (Kim et al., PNAS 93, 1156-1160 (1996); EGKSSGSGSESKVD (SEQ ID NO: 5) (Chaudhary et al., 1990, Proc. Natl. Acad. Sci. U.S.A. 87:1066-1070); KESGSVSSEQLAQFRSLD (SEQ ID NO: 6) (Bird et al., 1988, Science 242:423-426), GGRRGGGS (SEQ ID NO: 7); LRQRDGERP (SEQ ID NO: 8); LRQKDGGGSERP (SEQ ID NO: 9); LRQKd(GGGS)₂ ERP (SEQ ID NO: 10). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

In particular embodiments a CAR comprises a scFV that further comprises a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In one embodiment, the variable region linking sequence is 1, 2, 3, 4, 5, 6, 7, 8, 9, 10, 11, 12, 13, 14, 15, 16, 17, 18, 19, 20, 21, 22, 23, 24, 25, or more amino acids long. In a particular embodiment, the variable region linking sequence comprises a glycine-serine polymer (G₁₋₅S₁₋₅)ₙ, where n is an integer of at least 1, 2, 3, 4, or 5. In another embodiment, the variable region linking sequence comprises a (G₄S)₃ amino acid linker.

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 of IgG1.

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

An “altered hinge region” refers to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a naturally occurring immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Other illustrative hinge domains suitable for use in the CARs described herein include the hinge region derived from the extracellular regions of type 1 membrane proteins such as CD8α, CD4, CD28 and CD7, which may be wild-type hinge regions from these molecules or may be altered. In another embodiment, the hinge domain comprises a CD8a hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, and PD-1. In a particular embodiment, the TM domain is synthetic and predominantly comprises hydrophobic residues such as leucine and valine.

In one embodiment, the CARs contemplated herein comprise a TM domain derived from CD8α. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. A glycine-serine linker provides a particularly suitable linker.

In particular embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of the cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. In preferred embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are of particular use in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

CARs contemplated herein comprise one or more co-stimulatory signaling domains to enhance the efficacy and expansion of T cells expressing CAR receptors. As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory domains include isolated from TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70. In one embodiment, a CAR comprises one or more co-stimulatory signaling domains selected from the group consisting of CD28, CD137, and CD134, and a CD3ζ primary signaling domain.

In another embodiment, a CAR comprises CD28 and CD137 co-stimulatory signaling domains and a CD3ζ primary signaling domain.

In yet another embodiment, a CAR comprises CD28 and CD134 co-stimulatory signaling domains and a CD3ζ primary signaling domain.

In one embodiment, a CAR comprises CD137 and CD134 co-stimulatory signaling domains and a CD3ζ primary signaling domain.

In another embodiment, a CAR comprises CD28 co-stimulatory signaling domains and a CD3ζ primary signaling domain.

In yet another embodiment, a CAR comprises CD134 co-stimulatory signaling domains and a CD3ζ primary signaling domain.

In one embodiment, a CAR comprises CD137 co-stimulatory signaling domains and a CD3ζ primary signaling domain.

### H. Methods of Use

The compositions comprising trehalose and stabilized viral vectors contemplated herein may be used to provide gene therapy. As used herein, the term “gene therapy” refers to the introduction of a gene into a cell's genome. In various embodiments, compositions comprising stable viral vectors can be used for the transfer (and efficient integration) of heterologous DNAs (e.g., a therapeutic transgene) into eukaryotic cells. That is, recombinant viral vectors stabilized in trehalose containing compositions contemplated herein can be used as viral stock to infect recipient cells in culture or in vivo. In the case of secreted proteins or proteins expressed in hematopoietic cells, sensitive assays such as ELISA or Western blotting can be used to assess gene transfer efficiency.

Specifically, compositions comprising trehalose stabilized viral vectors can safely be used to transform not only a variety of dividing cell types, but also non-dividing cell types, increasing the range of diseases treatable by gene therapy.

In certain embodiments, the term “target cell” is used interchangeably with host cell and refers to transfected, infected, or transduced cells of a desired cell type.

A starting population of cells suitable for use in particular embodiments may be derived from essentially any suitable source, and may be heterogeneous or homogeneous with respect to cell types. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are allogeneic or autologous. Suitable cells include fetal cells and adult cells. In addition, suitable cells may be mammalian in origin, e.g., from a rodent, a cat, a dog, a pig, a goat, a sheep, a horse, a cow, or a primate. In one embodiment, the cells are human cells.

Exemplary host cells or target cells include, but are not limited to, stem cells, progenitor cells, and differentiated cells. Other suitable cells include, but are not limited to stem cells, progenitor cells, and differentiated cells. In certain embodiments, the transduced cells are embryonic stem cells, induced pluripotent stem cells, bone marrow stem cells, umbilical cord stem cells, placental stem cells, mesenchymal stem cells, neural stem cells, liver stem cells, pancreatic stem cells, pancreatic endoderm, cardiac stem cells, kidney stem cells and hematopoietic stem cells.

Other exemplary host cells or target cells include, but are not limited to, heterogeneous or homogeneous populations of cells selected from the group consisting of: pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like.

In particular embodiments, the cell is a hematopoietic cell, including but not limited to hematopoietic stem cells, CD34 expressing cells, hematopoietic progenitor cells, myeloid cells, lymphoid cells, B and T lymphocytes, and the like.

Viral vectors contemplated herein can be used in gene therapy, including for the treatment of hemoglobinopathies. In particular embodiments, methods for using the foregoing vectors to achieve stable, high levels of gene expression in erythroid cells, e.g., in order to treat erythroid-specific diseases are provided. In a particular embodiment, the gene therapy vectors are used to treat hemoglobinopathies, including, for example, sickle cell disease (SCD). In another preferred embodiment, the gene therapy vectors are used for treatment of thalassemias, including, but not limited to, β-thalassemia.

In another embodiment, hematopoietic stem cells are transduced with vectors contemplated herein comprising an ABCD1 gene for treatment of adrenoleukodystrophies and/or adrenomyeloneuropathies.

In other embodiments, viral vectors contemplated herein are also useful for the treatment of cancers, including, but not limited to, Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In various embodiments, the aqueous compositions comprising stable viral vectors are administered by direct injection to a cell, tissue, or organ of a subject in need of gene therapy, in vivo, e.g., intraosseous administration of genetically modified HSCs. In various other embodiments, cells are transduced in vitro or ex vivo with compositions comprising stable viral vectors, and optionally expanded ex vivo. The transduced cells are then administered to a subject in need of gene therapy.

In addition, the aqueous compositions comprising stable viral vectors can also be used to introduce DNA or genes of interest into mammalian cells, such as human cells, which will subsequently be administered into localized areas of the body (e.g., ex vivo infection of autologous white blood cells for delivery of protein into localized areas of the body, see e.g., U.S. Pat. No. 5,399,346).

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

The following examples show that trehalose and derivatives thereof are a suitable preservative for viral vectors. Viral vectors stored in solutions containing particular concentrations of trehalose or derivatives of trehalose maintain infectious titer in a broad range of temperatures and throughout multiple freeze thaw cycles compared to viral vectors not stored in trehalose or derivatives of trehalose.

### Example 1

### Materials and Methods

### Materials and Equipment

Human osteosarcoma (HOS) cells (ATCC, cat. no. CRL-1543)
DMEM high glucose (GIBCO, cat. no. 11995)
FBS, heat inactivated (GIBCO, cat. no. 26140-079)
Glutamax (GIBCO, cat. no. 35050)
6-well cell culture plates
Polystyrene round-bottomed tubes (5 mL, 12 mm×75 mm) (Becton Dickinson, cat. no. 352005)
Hank's balanced salt solution for flow cytometry. Mix 20 mL of 10× Hank's balanced salt solution with 4 mL of FBS and 176 mL of distilled water. This solution can be stored at 4° C. for at least 2 months.
Tissue culture hood
Tissue culture incubators
FACS machine

### Methods

HOS cells were plated at a density of 5×10⁴ cells per well in a 6-well plate 1 day before transduction.

Twenty-four hours after seeding, the number of cells in two of the wells were counted using a hemacytometer. The medium from other wells was removed and replaced with 0.5 mL of fresh medium containing 8 mg/mL of polybrene. Cells were transduced by adding 0.5-, 5- and 50-mL aliquots of concentrated vector stocks diluted 100-fold with cell culture medium (i.e., 1 μL of concentrated vector stock was mixed with 99 μL per well, respectively of medium). HOS cells were transduced by adding 0.5-, 5- and 50-mLaliquots, respectively, of the diluted vector suspension per well.

Twenty hours after beginning the transduction, the medium was replaced with 2 mL of fresh medium and continue to incubate cells.

After 2 days, the medium was removed and the cells were washed with 1 mL of PBS. Add 0.5 mL of trypsin-EDTA per well and incubate at 37° C. for 2 min. 1 mL of medium was added to each well, and the contents were mixed. The cell suspension from each well was transferred into a 5-mL round-bottomed tube and centrifuge at 1,500 r.p.m. (500 g) for 5 min at 20° C. to pellet cells.

The medium was removed by aspiration and cells pellets were resuspended in 2 mL of Hank's balanced salt solution and centrifuged at 1,500 r.p.m. (500 g) for 5 min at 20° C. to pellet cells.

Hank's balanced salt solution was removed by aspiration and cells pellets were resuspended in 300 mL of Hank's balanced salt solution.

The cells were then analyzed by FACS.

The titer (transducing units (TU)/mL) was calculated according to the following formula: TU/mL=(F×N×D×1,000)/V, where F=percentage of fluorescent cells (EGFP), N=number of cells at the time of transduction (corresponding to about 1E+05 HOS cells per well), D=fold dilution of vector sample used for transduction and V=volume (mL) of diluted vector sample added into each well for transduction. Vector titers obtained from HOS cells transduced with different amounts of vector were combined and an average titer was calculated. For accurate titer determination, the amount of vector used should fall in a range in which there is a linear relationship between the percentage of EGFP-positive cells and the amount of vector added. If the percentage of fluorescent cells exceeds 40%, the titration is repeated using additional vector dilutions.

### Example 2

### Long-Term Stability of Viral Vectors Stored in Trehalose

Lentiviral vectors were produced and purified using standard methods. Final vector products were purified by size exclusion chromatography and either stored undiluted in phosphate buffered saline pH 7.4 (PBS) or diluted in 5% trehalose/PBS (Sigma-Aldrich Cat#90210).

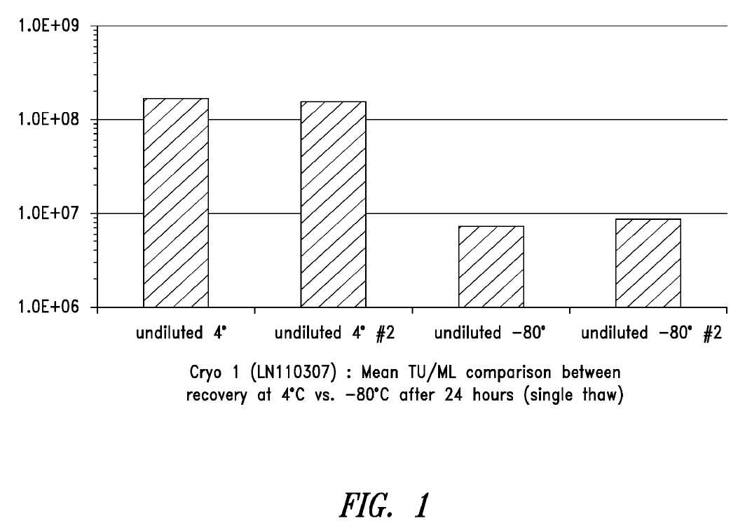

The effect of thawing on lentiviral vector titer was examined in undiluted lentiviral vector compositions stored for 24 hours at 4° C. and after a single thaw when stored at −80° C. for 24 hours. FIG. 1 shows that the mean titer (TU/mL) was reduced in undiluted lentiviral vector compositions stored at −80° C. compared to undiluted lentiviral vector stored at 4° C.

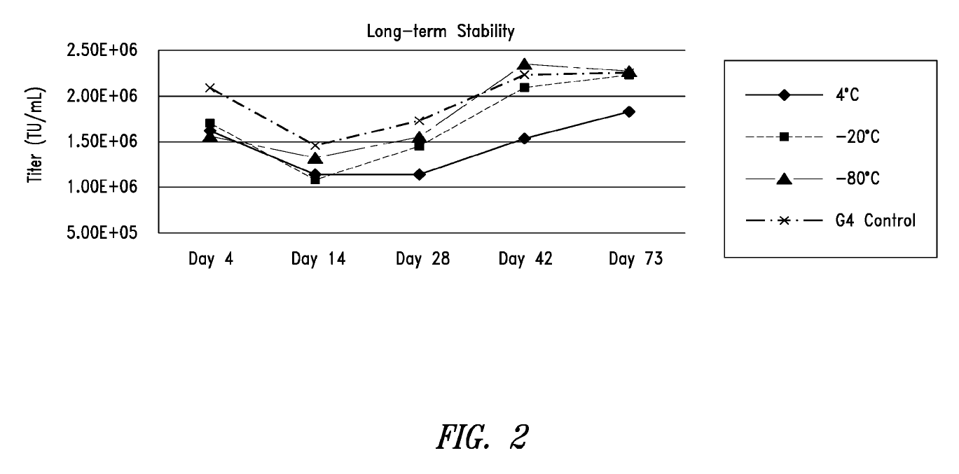

Next, the long-term stability of lentiviral vectors stored undiluted in PBS or diluted in 5% trehalose/PBS was examined over various temperatures for extended periods of storage. Titer was measured for lentiviral vectors stored for 4, 14, 28, 42, and 73 days at 4° C. and at −20° C. and −80° C. after single thaws. FIG. 2 shows that lentiviral vectors formulated in 5% trehalose show stable lentiviral titer across the temperatures tested for at least 73 days.

### Example 3

### Stabilization of Viral Vectors in Trehalose

Lentiviral vectors expressing a red fluorescent protein (RFP) were produced and purified using standard methods. Final vector products were purified by size exclusion chromatography and either stored undiluted in PBS or diluted in about 7.5% trehalose/PBS, about 15% trehalose/PBS, about 18.75% trehalose/PBS, and about 22.7% trehalose/PBS.

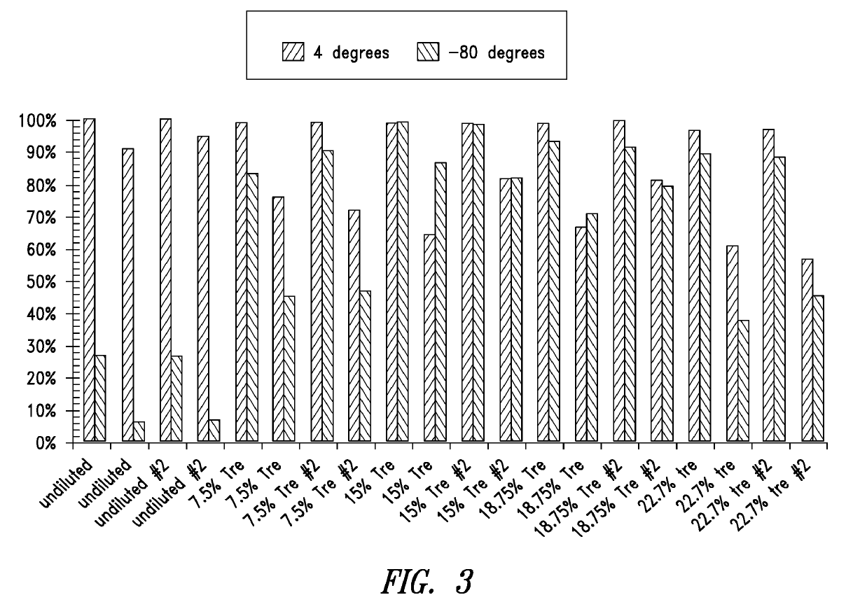

Lentiviral vector expression (RFP expression) was examined by flow cytometry in cells transduced with 1E+08 TU/mL formulated in various concentrations of trehalose. FIG. 3 shows that greater percentages of cells containing the lentiviral vector expressed RFP when stored for 24 hours at 4° C. and after a single thaw when stored at −80° C. for 24 hours in trehalose/PBS compared to cells containing the lentiviral vector stored undiluted in PBS. The greatest percentage of cells expressing RFP occurred when cells were stored in about 15% trehalose/PBS, reaching from about 98% to about 99% cells expressing RFP at both 4° C. and −80° C.

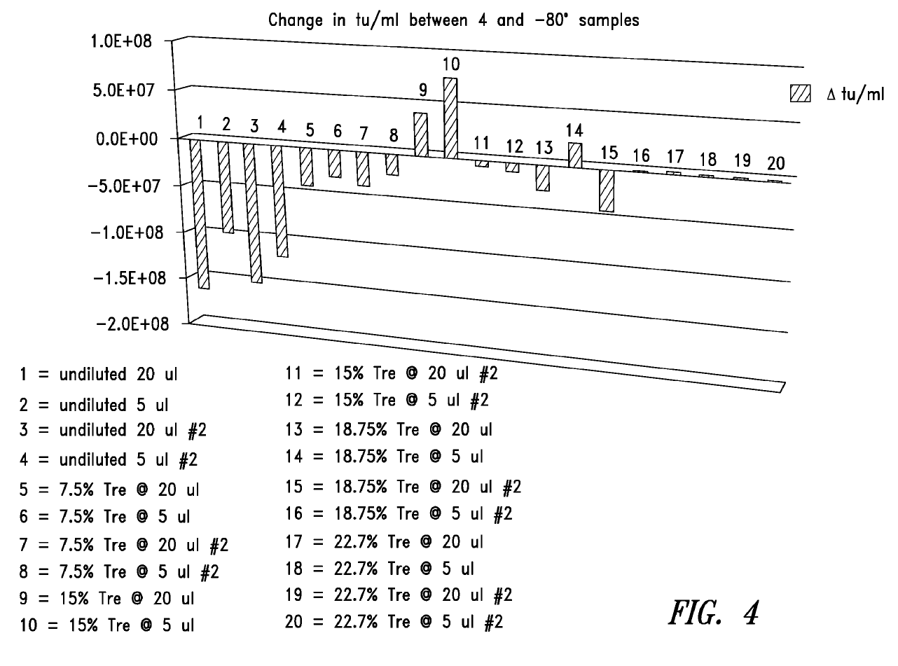

The change in mean titer (TU/mL) for these same cell samples was examined to determine which storage conditions provided the most protection for the lentiviral vector when stored at the lower temperature of −80° C. compared to when stored at 4° C. As seen in FIG. 4, cells containing the lentiviral vector stored undiluted in PBS had large negative Δ TU/mL values, indicating that the lentiviral vector did not retain stability once stored at −80° C. for 24 hours and then thawed compared to storage at 4° C. Consistent with the flow cytometry data, cells containing the lentiviral vector stored in about 15% trehalose/PBS showed the largest positive Δ TU/mL values, showing that the mean titers for the lentiviral vector actually increased after storage at −80° C. for 24 hours and then thawed compared to storage at 4° C.

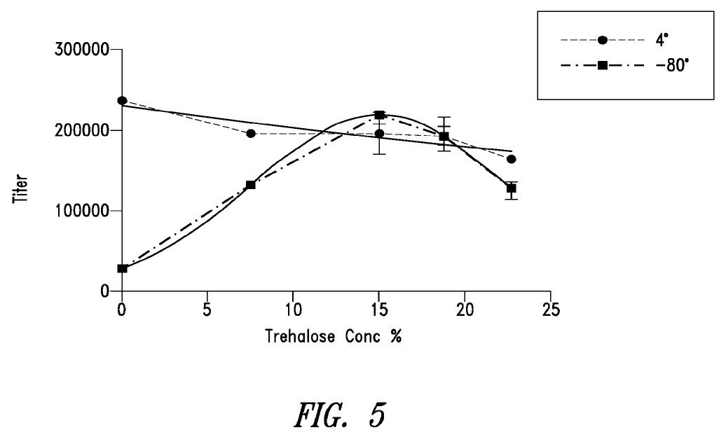

When the titer of the lentiviral vector was plotted against the trehalose concentration (% by weight) of the storage composition (FIG. 5), the percentage of trehalose that yielded the highest preservation of the lentiviral vector with the lowest dilution at both 4° C. and −80° C. was about 15%, again consistent with results obtained for the vector in the flow cytometry experiments and in the calculation of Δ TU/mL values between 4° C. and −80° C. storage.

### Example 4

### Stabilization of Viral Vectors in Small Quantities of Trehalose

Lentiviral vectors expressing a green fluorescent protein (GFP) were produced and purified using standard methods. Final vector products were purified by size exclusion chromatography and either stored undiluted in PBS, diluted 50% in dPBS or diluted in about 5.67% trehalose/PBS, about 6.615% trehalose/PBS and about 7.56% trehalose/PBS.

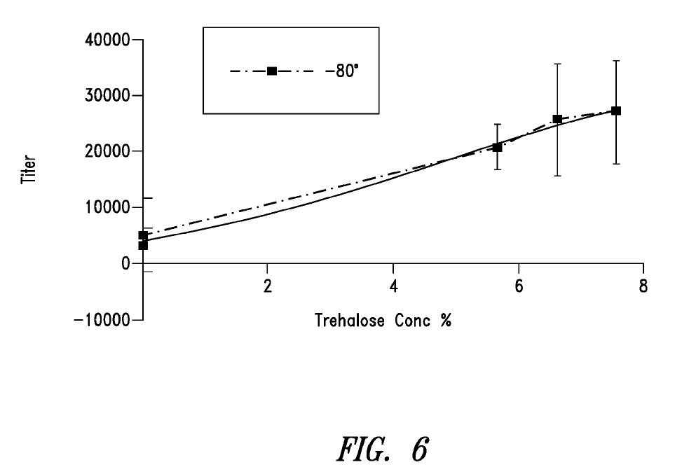

The titer of the lentiviral vectors was examined for samples stored at −80° C. in various concentrations of trehalose/PBS. For either concentrated product undiluted or diluted 1:2 in dPBS, the preservative effect of trehalose was linear at the lower trehalose concentrations tested (FIG. 6). This suggests the possibility that trehalose even in small quantities exhibits linear preservative effects.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An aqueous composition comprising:
(a) a viral vector;
(b) about 3% to about 15% trehalose or derivatives thereof by weight; and
(c) a pharmaceutically acceptable diluent.

2. The composition of claim 1, wherein the vector is an adenoviral vector, an adeno-associated viral vector, or a retroviral vector.

3. The composition of claim 1, wherein the viral vector is a retroviral vector.

4. The composition of claim 1, wherein the viral vector is a lentiviral vector.

5. The composition of claim 4, wherein the lentiviral vector is selected from the group consisting essentially of human immunodeficiency virus (HIV); visna-maedi virus (VMV); caprine arthritis-encephalitis virus (CAEV); equine infectious anemia virus (EIAV); feline immunodeficiency virus (Hy); bovine immune deficiency virus (BIV); and simian immunodeficiency virus (SIV).

6. The composition of claim 4, wherein the lentiviral vector is HIV-1.

7. The composition of claim 4, wherein the lentiviral vector is HIV-2.

8. The composition of claim 1, wherein the viral vector comprises an envelope polypeptide of a virus selected from the group consisting of: Avian leukosis virus (ALV), FIV, HIV, vesicular stomatitis virus (VSV), moloney murine leukemia virus (MoMLV), gibbon ape leukemia virus (GaLV), jaagsiekte sheep retrovirus (JSRV), lymphocytic choriomeningitis virus (LCMV), Human T-lymphotropic virus 1 (HTLV-1), visna-maedi virus (VMV), SARS-CoV, Chandipura virus, Marburg virus, Mokola virus, feline endogenous retrovirus (RD114), Ebola virus, Rabies virus, Ross River virus (RRV), Respiratory syncytia virus (RSV), Human parainfluenza virus type 3, Hepatitis C virus (HCV), Sendai virus, Sindbis virus, Semliki Forest virus (SFV), foul plague virus (FPV), influenza virus, Venezuelan equine encephalitis virus, and Lagos-bat virus.

9. The composition of claim 8, wherein the viral vector comprises the envelope polypeptide from VSV or RD114.

10. The composition of claim 1, wherein the viral vector comprises a polynucleotide sequence encoding a chimeric antigen receptor (CAR) polypeptide, an engineered T cell receptor polypeptide, or a bi-specific T cell engager polypeptide.

11. The composition of claim 10, wherein the CAR comprises:
a) an extracellular domain that binds an antigen selected from the group consisting of: alpha folate receptor, 5T4, αvβ6 integrin, BCMA, B7-H3, B7-H6, CAIX, CD16, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAIVIE, PSCA, PSMA, ROR1, SSX, Survivin, TAG72, TEMs, VEGFR2, and WT-1;
b) a transmembrane domain derived from a polypeptide selected from the group consisting of: CD8α, CD4, CD28, CD45, PD1, CTLA-4, and CD152;
c) one or more intracellular co-stimulatory signaling domains selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and
d) a CD3ζ primary signaling domain.

12. The composition of claim 11, wherein the extracellular domain comprises an antibody or antigen binding fragment that binds the antigen.

13. The composition of claim 11, wherein the antibody or antigen binding fragment that binds the BCMA polypeptide is selected from the group consisting of: a Camel Ig, Ig NAR, Fab fragments, Fab′ fragments, F(ab)′₂ fragments, F(ab)′₃ fragments, Fv, single chain Fv antibody (“scFv”), bis-scFv, (scFv)₂, minibody, diabody, triabody, tetrabody, disulfide stabilized Fv protein (“dsFv”), and single-domain antibody (sdAb, Nanobody).

14. The composition of claim 13, wherein the antibody or antigen binding fragment that binds the BCMA polypeptide is an scFv.

15. The composition of any one of claims 11 to 13, wherein the antibody is a human antibody, a murine antibody, or a humanized antibody.

16. The composition of claim 11, wherein the transmembrane domain is derived from CD8α.

17. The composition of claim 11, wherein the one or more intracellular co-stimulatory signaling domains is selected from the group consisting of: CD28, CD134, and CD137.

18. The composition of claim 11, wherein the CAR comprises two or more intracellular co-stimulatory signaling domains selected from the group consisting of: CD28, CD134, and CD137.

19. The composition of claim 11, wherein the one or more intracellular co-stimulatory signaling domains is CD28.

20. The composition of claim 11, wherein the one or more intracellular co-stimulatory signaling domains is CD134.

21. The composition of claim 11, wherein the one or more intracellular co-stimulatory signaling domains is CD137.

22. The composition of claim 11, further comprising a hinge region polypeptide.

23. The composition of claim 22, wherein the hinge region polypeptide comprises a hinge region of CD8α.

24. The composition of any one of claims 11 to 23, further comprising a spacer region polypeptide.

25. The composition of claim 24, wherein the spacer region polypeptide comprises CH2 and CH3 regions of IgG1.

26. The composition of any one of claims 11 to 25, further comprising a signal peptide.

27. The composition of claim 26, wherein the signal peptide comprises an IgG1 heavy chain signal polypeptide or a CD8α signal polypeptide.

28. The composition of claim 1, wherein the viral vector comprises a polynucleotide sequence encoding a homing endonuclease, a transcription activator-like effector nuclease (TALEN), a zinc finger nuclease (ZFN), a Type II clustered regularly interspaced short palindromic repeats (CRISPR) associated (Cas9) nuclease, or a megaTAL nuclease.

29. The composition of claim 1, wherein the viral vector comprises a polynucleotide sequence encoding a β-globin polypeptide.

30. The composition of claim 1, wherein the viral vector comprises a polynucleotide sequence encoding an ABCD1 polypeptide.

31. The composition of claim 1, wherein the composition comprises about 3% trehalose or derivatives thereof by weight.

32. The composition of claim 1, wherein the composition comprises about 4% trehalose or derivatives thereof by weight.

33. The composition of claim 1, wherein the composition comprises about 5% trehalose or derivatives thereof by weight.

34. The composition of claim 1, wherein the composition comprises about 6% trehalose or derivatives thereof by weight.

35. The composition of claim 1, wherein the composition comprises about 7% trehalose or derivatives thereof by weight.

36. The composition of claim 1, wherein the composition comprises about 8% trehalose or derivatives thereof by weight.

37. The composition of claim 1, wherein the composition comprises about 9% trehalose or derivatives thereof by weight.

38. The composition of claim 1, wherein the composition comprises about 10% trehalose or derivatives thereof by weight.

39. The composition of claim 1, wherein the composition comprises about 11% trehalose or derivatives thereof by weight.

40. The composition of claim 1, wherein the composition comprises about 12% trehalose or derivatives thereof by weight.

41. The composition of claim 1, wherein the composition comprises about 13% trehalose or derivatives thereof by weight.

42. The composition of claim 1, wherein the composition comprises about 14% trehalose or derivatives thereof by weight.

43. The composition of claim 1, wherein the composition comprises about 15% trehalose or derivatives thereof by weight.

44. The composition of claim 1, wherein the composition comprises about 5% to about 15% trehalose or derivatives thereof by weight.

45. The composition of claim 1, wherein the composition comprises about 4% to about 12% trehalose or derivatives thereof by weight.

46. The composition of claim 1, wherein the composition comprises about 5% to about 10% trehalose or derivatives thereof by weight.

47. The composition of claim 1, wherein the composition comprises about 5% to about 9% trehalose or derivatives thereof by weight.

48. The composition of claim 1, wherein the composition comprises about 5% to about 8% trehalose or derivatives thereof by weight.

49. The composition of claim 1, wherein the composition comprises about 5% to about 7% trehalose or derivatives thereof by weight.

50. The composition of claim 1, wherein the composition comprises about 4% to about 6% trehalose or derivatives thereof by weight.

51. The composition of claim 1, wherein the pharmaceutically acceptable diluent comprises a physiologically acceptable buffer.

52. The composition of claim 51, wherein the physiologically acceptable buffer is selected from the group consisting of: Hanks buffered saline solution (HBSS), Ringer's solution, Dulbecco's phosphate buffered saline (PBS), 5% dextrose in water (D5W), and physiologic saline (0.9% NaCl).

53. The composition of claim 1, wherein the pharmaceutically acceptable diluent comprises a physiologically acceptable cell culture medium.

54. The composition of claim 53, wherein the pharmaceutically acceptable cell culture medium is selected from the group consisting of: StemSpan-ACF, StemSpan-H3000, StemSpan-SFEM, Stemline II, StemPro 34, StemXVivo, Iscove's modified Dulbecco's medium (IMDM), Dulbecco's modified Eagle medium (DMEM), Roswell Park Memorial Institute medium (RPMI) 1640 medium, McCoy's 5A medium, minimum essential medium alpha medium (alpha-MEM), basal medium Eagle (BME), Fischer's medium, medium199, F-12K nutrient mixture medium (Kaighn's modification, F-12K), and X-vivo 20.

55. The composition of any one of claims 1 to 54, wherein the viral vector has a titer that is stable for more than one month when the composition is stored at about 20° C.

56. The composition of any one of claims 1 to 54, wherein the viral vector has a titer that is stable for more than six months when the composition is stored at about 20° C.

57. The composition of any one of claims 1 to 54, wherein the viral vector has a titer that is stable for more than one year when the composition is stored at about 20° C.

58. The composition of any one of claims 1 to 54, wherein the viral vector has a titer that is stable for more than one month when the composition is stored at about 10° C.

59. The composition of any one of claims 1 to 54, wherein the viral vector has a titer that is stable for more than six months when the composition is stored at about 10° C.

60. The composition of any one of claims 1 to 54, wherein the viral vector has a titer that is stable for more than one year when the composition is stored at about 10° C.

61. The composition of any one of claims 1 to 54, wherein the viral vector has a titer that is stable for more than one month when the composition is stored at about 4° C.

62. The composition of any one of claims 1 to 54, wherein the viral vector has a titer that is stable for more than six months when the composition is stored at about 4° C.

63. The composition of any one of claims 1 to 54, wherein the viral vector has a titer that is stable for more than one year when the composition is stored at about 4° C.

64. The composition of any one of claims 1 to 54, wherein the viral vector has a titer that is stable for more than one year when the composition is stored at about 0° C.

65. The composition of any one of claims 1 to 54, wherein the viral vector has a titer that is stable for more than one year when the composition is stored at about −20° C.

66. The composition of any one of claims 1 to 54, wherein the viral vector has a titer that is stable for more than one year when the composition is stored at about −60° C.

67. The composition of any one of claims 1 to 54, wherein the viral vector has a titer that is stable for more than one year when the composition is stored at about −70° C.

68. The composition of any one of claims 1 to 54, wherein the viral vector has a titer that is stable for more than one year when the composition is stored at about −80° C.

69. The composition of any one of claims 1 to 68, wherein the titer of the viral vector is stable for one or more freeze/thaw cycles.

70. The composition of any one of claims 1 to 68, wherein the titer of the viral vector is stable for two or more freeze/thaw cycles.

71. The composition of any one of claims 1 to 68, wherein the titer of the viral vector is stable for three or more freeze/thaw cycles.

72. The composition of any one of claims 1 to 71, wherein the composition is suitable for direct in vivo injection.

73. The composition of any one of claims 1 to 71, wherein the composition is suitable for direct in vitro use.

74. The composition of claim 72 or claim 73, wherein the composition is diluted at least 10× prior to direct in vivo injection or direct in vitro use.

75. The composition of claim 72 or claim 73, wherein the composition is diluted at least 50× prior to direct in vivo injection or direct in vitro use.

76. The composition of claim 72 or claim 73, wherein the composition is diluted at least 100× prior to direct in vivo injection or direct in vitro use.

77. The composition of claim 72 or claim 73, wherein the composition is diluted at least 200× prior to direct in vivo injection or direct in vitro use.

78. The composition of claim 72 or claim 73, wherein the composition is diluted at least 250× prior to direct in vivo injection or direct in vitro use.

79. A method of treating a disease in a subject in need of treatment comprising administering to said subject a population of cells transduced with a composition of claim 1.

80. A method of transducing a cell with a composition of claim 1 comprising introducing the composition of any one of claims 1-79 into a population of cells.

81. The method of claim 80, wherein the cell is a mammalian cell.

82. The method of claim 80, wherein the cell is a human cell.

83. The method of claim 80, wherein the cell is a stem cell or progenitor cell.

84. The method of claim 80, wherein the cell is a hematopoietic cell.

85. The method of claim 84, wherein the hematopoietic cell is a hematopoietic stem or progenitor cell or a cell that expresses CD34.

86. The method of claim 84, wherein the hematopoietic cell is a lymphocyte.

87. The method of claim 86, wherein the lymphocyte is a T lymphocyte.

88. A method for stabilizing viral vectors comprising:
(a) preparing a composition comprising trehalose or derivatives thereof; and
(b) adding a viral vector to the composition.

89. The method of claim 81, wherein the viral vector has a titer that is stable when stored at a temperature range of about −80° C. to about 25° C. for at least one year.

90. The method of claim 81, wherein the viral vector has a titer that is stable when stored at a temperature range of about −20° C. to about 18° C. for at least one year.

91. The method of claim 81, wherein the viral vector has a titer that is stable when stored at a temperature range of about −20° C. to about 4° C. for at least one year.

92. The method of claim 81, wherein the viral vector has a titer that is stable when stored at a temperature range of about 4° C. to about 25° C. for at least one year.

93. The method of claim 81, wherein the viral vector has a titer that is stable when stored at a temperature range of about 4° C. to about 18° C. for at least one year.


SEQUENCE LISTING (10 sequences)

SEQ ID NO: 1 - protein, 5 aa, Artificial Sequence
  CAR linker sequence
DGGGS

SEQ ID NO: 2 - protein, 5 aa, Artificial Sequence
  CAR linker sequence
TGEKP

SEQ ID NO: 3 - protein, 4 aa, Artificial Sequence
  CAR linker sequence
GGRR

SEQ ID NO: 4 - protein, 5 aa, Artificial Sequence
  CAR linker sequence
GGGGS

SEQ ID NO: 5 - protein, 14 aa, Artificial Sequence
  CAR linker sequence
EGKSSGSGSESKVD

SEQ ID NO: 6 - protein, 18 aa, Artificial Sequence
  CAR linker sequence
KESGSVSSEQLAQFRSLD

SEQ ID NO: 7 - protein, 8 aa, Artificial Sequence
  CAR linker sequence
GGRRGGGS

SEQ ID NO: 8 - protein, 9 aa, Artificial Sequence
  CAR linker sequence
LRQRDGERP

SEQ ID NO: 9 - protein, 12 aa, Artificial Sequence
  CAR linker sequence
LRQKDGGGSERP

SEQ ID NO: 10 - protein, 16 aa, Artificial Sequence
  CAR linker sequence
LRQKDGGGSGGGSERP